(19) United States
(12) Patent Application Publication
(10) Pub. No.: US 2023/0015056 A1
(43) Pub. Date: Jan. 19, 2023

(54) DISCHARGE RISK AND MANAGEMENT

(71) Applicant: Hill-Rom Services, Inc., Batesville, IN (US)

(72) Inventors: Dee Anna Kumpar, Freeland, MI (US); Neal E. Wiggermann, Batesville, IN (US); Karrie Ann Schwencer, Cary, NC (US); Eugene G. Urrutia, Apex, NC (US)

(21) Appl. No.: 17/812,452

(22) Filed: Jul. 14, 2022

Related U.S. Application Data
(60) Provisional application No. 63/222,222, filed on Jul. 15, 2021.
(60) Provisional application No. 63/297,144, filed on Jan. 6, 2022.

(51) Int. Cl. G16H 40/20; G16H 10/60

(52) U.S. Cl. CPC G16H 40/20; G16H 10/60

(57) ABSTRACT

A method comprising receiving an input indicating intake information associated with a patient. Based on the input, the method further includes determining an initial discharge date and receiving mobility information associated with the patient. Based in part on the mobility information, the method further includes determining an estimated discharge date and a confidence metric associated with the estimated discharge date, determining that the estimated discharge date is later than the initial discharge date by more than a threshold period of time, and determining that the confidence metric is greater than a threshold metric. Based in part on the estimated discharge date being later than the initial discharge date by more than the threshold period of time and the confidence metric being greater than the threshold metric, the method further includes generating an alert.

### CROSS REFERENCE TO RELATED APPLICATIONS

This application is a Nonprovisional of, and claims priority to, U.S. Provisional Patent Application No. 63/297,144, filed Jan. 6, 2022, and claims priority to, U.S. Provisional Patent Application No. 63/222,222, filed Jul. 15, 2021, the entire disclosures of which are incorporated herein by reference.

### TECHNICAL FIELD

The present application relates to systems and methods for monitoring and managing discharge risk.

### BACKGROUND

A patient's mobility impacts the equipment, staffing, and care protocols that are selected for the patient within a healthcare facility such as a hospital. Additionally, a patient's mobility can help determine whether the patient can be safely discharged from the healthcare facility. For instance, medically complex patients with long lengths of stay at a hospital are typically costly to hospitals and may contribute to bottom-line losses. For some patients, these extended stays are predictable and unavoidable under the current practice. For other patients, these extended stays are due to unanticipated functional decline, which may result in a “day of discharge surprise.”

A “day of discharge surprise” most commonly occurs for patients who experience a decline in mobility during their hospital admission that goes undocumented or uncommunicated. In some cases, the decline in mobility is secondary to the condition for admission, such that the original discharge goal cannot be met even though the primary condition is treated and discharge is expected. Accordingly, a “day of discharge surprise” can be costly. First, the hospital is unable to discharge the patient in a timely manner and the extended stay is likely not to receive additional reimbursement, resulting in the additional costs being absorbed by the hospital. In some cases, such as if a particular unit is full or the patient is using specialized resources, the hospital may not have additional equipment or staffing to compensate for the additional time the patient is staying. Moreover, discharge planners and physical therapists at the hospital may suddenly have an emergent problem that shifts their focus to find an appropriate place for the patient. Additionally, the patient may receive the unwanted surprise of a delay in discharge and/or be discharged to an unexpected location (e.g., such as rehabilitation facility) instead of to the patient's home.

Examples of the present disclosure are directed toward overcoming the issues noted above.

### SUMMARY

In an example of the present disclosure, a method comprises receiving an input indicating intake information associated with a patient, determining, based on the input, an initial discharge date, receiving mobility information associated with the patient, determining, based at least partly on the mobility information, an estimated discharge date and a confidence metric associated with the estimated discharge date, determining that the estimated discharge date is later than the initial discharge date by more than a threshold period of time, determining that the confidence metric is greater than a threshold metric, and based at least partly on the estimated discharge date being later than the initial discharge date by more than the threshold period of time and the confidence metric being greater than the threshold metric, generating an alert.

In an example of the present disclosure, a system comprises a network, a computing device connected to the network, a controller in communication with the computing device via the network, a plurality of sensors operably connected to the controller via the network, and one or more non-transitory computer-readable media. The one or more non-transitory computer-readable media can store instructions that, when executed by the controller, cause the controller to perform operations comprising: receive, via the network and from the computing device, an input indicating intake information associated with a patient, determine, based on the input, an initial discharge date; receive, via the network and from the plurality of sensors, mobility information associated with the patient, determine, based at least partly on the mobility information, an estimated discharge date and a confidence metric associated with the estimated discharge date, determine that the estimated discharge date is later than the initial discharge date by more than a threshold period of time, determine that the confidence metric is greater than a threshold metric, generate an alert based at least partly on the estimated discharge date being later than the initial discharge date by more than the threshold period of time and the confidence metric being greater than the threshold metric, and provide, via the network, the alert to the computing device, the alert causing the computing device to display at least one of a visual indicia or a suggestion in response to receiving the alert.

In yet another example of the present disclosure, a method comprises determining, by a controller, one or more of an initial discharge date, a mobility requirement, or a discharge event, receiving, by the controller, mobility information associated with a patient, determining, by the controller and based at least partly on the mobility information, an estimated discharge date and a confidence metric associated with the estimated discharge date, determining, by the controller, that at least one of the mobility requirement or the discharge event is achievable by a date that is later than the initial discharge date, determining, by the controller, that the confidence metric is greater than a threshold metric, and generating, by the controller, an alert.

The details of one or more embodiments are set forth in the accompanying drawings and the description below. Other features, objects, and advantages of these embodiments will be apparent from the description, drawings, and claims.

### DESCRIPTION OF THE FIGURES

The present invention may comprise one or more of the features recited in the appended claims and/or one or more of the following features or combinations thereof. Additionally, in this specification and drawings, features similar to or the same as features already described may be identified by reference characters or numerals which are the same as or similar to those previously used. Similar elements may be identified by a common reference character or numeral, with suffixes being used to refer to specific occurrences of the element.

FIG. 1 shows a schematic block diagram of an example discharge management system.

FIG. 2 illustrates an example patient support apparatus associated with the example discharge management tracking system of FIG. 1.

FIG. 3 illustrates an example of a walk assist device associated with the example mobility management system of FIG. 1.

FIG. 4 illustrates an example method associated with the example discharge management system of FIGS. 1-3.

FIG. 5 illustrates another example method associated with the example discharge management system of FIGS. 1-4.

FIG. 6 illustrates a schematically illustrates an example controller associated with the example discharge management system of FIG. 1.

### DETAILED DESCRIPTION

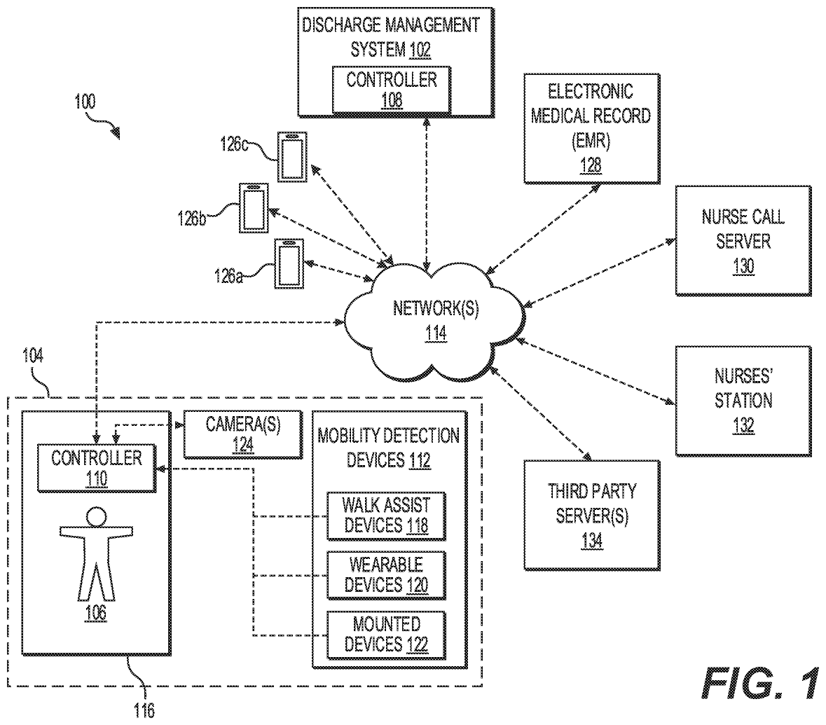

FIG. 1 is a schematic block diagram of an example system 100 for a discharge management system 102. In some examples, the discharge management system 102 comprises one or more servers, computing devices, and/or a cloud-based system. In some examples, the discharge management system 102 is located within a healthcare facility. In other examples, the discharge management system 102 is located remote from a healthcare facility.

As illustrated in FIG. 1, the patient environment 104 is a patient room within a healthcare facility such as a hospital, a surgical center, a nursing home, a long-term care facility, and the like. In other examples, the patient environment 104 is the home of the patient 106.

As illustrated in FIG. 1, the discharge management system 102 comprises a controller 108. Controller 108 comprises an electronic controller that operates in a logical fashion to perform operations, execute control algorithms, store and retrieve data, and other desired operations. The controller 108 may include and/or access memory, secondary storage devices, processors, and any other components for running an application. The memory and secondary storage devices may be in the form of read-only memory (ROM) or random-access memory (RAM) or integrated circuitry that is accessible by the controller. Various other circuits may be associated with the system controller 108 such as power supply circuitry, signal conditioning circuitry, driver circuitry, and other types of circuitry.

Controller 108 may be a single controller or may include more than one controller. In examples where the controller 108 includes more than one controller, the controller 108 may, for example, include additional controllers (e.g., such as controller 110) configured to control various functions and/or features of the system 100. As used herein, the term “controller” is meant in its broadest sense to include one or more controllers, processors, central processing units, and/or microprocessors that may be associated with the system 100, and that may cooperate in controlling various functions and operations of the system 100. The functionality of the controller 108 may be implemented in hardware and/or software without regard to the functionality. The controller 108 may rely on one or more data maps, look-up tables, neural networks, algorithms, machine learning algorithms, and/or other components relating to the operating conditions and the operating environment of the system 100 that may be stored in the memory of the controller 108. Each of the data maps, look-up tables, neural networks, and/or other components noted above may include a collection of data in the form of tables, graphs, and/or equations to maximize the performance and efficiency of the system 100 and its operation.

As illustrated in FIG. 1, the patient environment 104 includes controller 110. In some examples, the controller 110 comprises an electronic controller that operates in a logical fashion to perform operations, execute control algorithms, store and retrieve data, and other desired operations. The controller 110 may include and/or access memory, secondary storage devices, processors, and any other components for running an application. The memory and secondary storage devices may be in the form of read-only memory (ROM) or random-access memory (RAM) or integrated circuitry that is accessible by the controller. Various other circuits may be associated with the system controller 108 such as power supply circuitry, signal conditioning circuitry, driver circuitry, and other types of circuitry. In some examples, controller 110 comprises a centralized analytics computation device within the patient environment 104.

Controller 110 may be a single controller or may include more than one controller. In examples where the controller 110 includes more than one controller, the controller 110 may, for example, include additional controllers (e.g., such as controller 108) configured to control various functions and/or features of the system 100. The functionality of the controller 110 may be implemented in hardware and/or software without regard to the functionality. The controller 110 may rely on one or more data maps, look-up tables, neural networks, algorithms, machine learning algorithms, and/or other components relating to the operating conditions and the operating environment of the system 100 that may be stored in the memory of the controller 110. Each of the data maps, look-up tables, neural networks, and/or other components noted above may include a collection of data in the form of tables, graphs, and/or equations to maximize the performance and efficiency of the system 100 and its operation.

In some examples, controller 108 and/or controller 110 receives movement data from mobility detection devices 112 and/or patient support apparatus 116 and aggregates the movement data detected by the mobility detection devices 112 with the movements detected by the patient support apparatus 116. In some examples, by implementing the processing of the movement data, sensor data, and other data by the controller 108, the method described herein reduces the amount of data that is transmitted across a local area network (e.g., such as network(s) 114) of a healthcare facility, and/or reduces the amount of data that is processed and analyzed by on-premises or edge-servers of the healthcare facility. This, in turn. reduces the strain on the bandwidth of the local area network and on-premises servers. In some examples, the processing performed by the controller 108 and/or controller 110 improves the data upload bandwidth and reduces network lags for cloud-based servers utilized by the healthcare facility to perform data processing and analytics.

Network(s) 114 comprise any type of wireless network or other communication network known in the art. In some examples, the network 114 comprises a local area network (“LAN”), a WiFi direct network, wireless LAN (“WLAN”), personal area network (“PAN”), virtual PAN (“VPAN”), a larger network such as a wide area network (“WAN”), cellular network connections, or a collection of networks, such as the Internet. Protocols for network communication, such as TCP/IP, 802.11a, b, g, n and/or ac, are used to implement the network 114. Although embodiments are described herein as using a network 114 such as the Internet, other distribution techniques may be implemented that transmit information via memory cards, flash memory, or other portable memory devices.

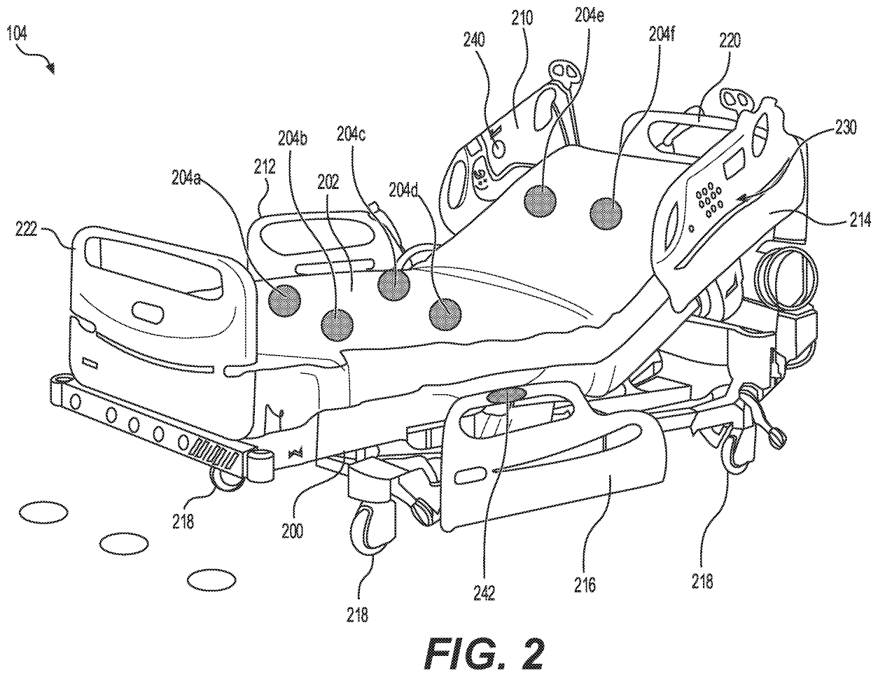

As shown in FIG. 1, the patient environment 104 includes a patient support apparatus 116 on which the patient 106 rests. In some examples, the patient support apparatus 116 comprises a bed, such as the one shown and described in greater detail below with reference to FIG. 2. In some examples, the patient support apparatus 116 comprises be a chair, a recliner, a stretcher, surgical table, or any other apparatus on which a patient can rest. As will be described in greater detail below, the patient support apparatus 116 includes sensors that detect movements of the patient 106 while the patient 106 rests on the patient support apparatus 116.

Patient environment 104 further includes mobility detection devices 112. Mobility detection devices 112 each comprise one or more sensors that detect the movements of the patient 106 when the patient 106 is not resting on the patient support apparatus 116. The mobility detection devices 112 include walk assist devices 118, wearable devices 120, and mounted devices 122. In some examples, the camera(s) 110 are included as part of the mobility detection devices 112.

Each mobility detection device 112 is configured to communicate the detected movements of the patient 106 to controller 108 and/or controller 110. In the example illustrated in FIG. 1, controller 110 is a component of the patient support apparatus 116, and may also be used to control the operation of the patient support apparatus 116. In some examples, the controller 110 is a standalone device (e.g., such as a patient monitor, nursing station monitor, etc.) external from the patient support apparatus 116. In other examples, the controller 110 comprises a component of another device or apparatus located in the patient environment 104.

In addition to detecting movements of the patient 106, each mobility detection device 112, such as via the sensors, detects and transmits additional parameters to the controller 108 and/or controller 110 including patient vital signs such as heart rate, respiration rate, SpO₂, and non-invasive blood pressure. The detected movements and additional parameters of the patient 106 are used by the controller 108 to determine a variety of clinical assessments and scores, including a Braden's mobility score, sepsis prediction score, a pressure injury risk score, a falls risk score, and the like.

In some examples, the sensor(s) of the patient support apparatus 116 and/or mobility detection devices 112 are communicatively connected to the controller 108 and/or controller 110 via one or more wireless communication protocols. In some examples, the one or more wireless communication protocols include Wi-Fi, Bluetooth, Z-Wave, Zigbee, and the like, and/or one or more wired communications protocols including Ethernet, USB, and like. In some examples, the one or more wireless communication protocols provide an Internet of Things (IOT) network within the patient environment 104 that is private and secure. This can improve communication among the devices located within the patient environment 104, to authenticate, authorize, and associate the devices with each other, and perform coordinated operations. These functionalities, including authentication and authorization, can be distributed and performed in the IOT network with or without using a dedicated edge-computational resources of the healthcare facility. In some examples, the patient support apparatus 116 and/or mobility detection devices 112 may act as a communication and computation network node within the IOT network. In some examples, the functionalities are localized at the controller 108 and/or controller 110 and/or distributed among network nodes using or completely avoiding an edge or cloud based computational infrastructure. The IOT network enables the controller 108 and/or controller 110 to locally and remotely monitor and control each or some of the mobility detection devices 112 and the patient support apparatus 116 through device or caregiver issued instructions. Additionally, the controllers 108 and/or 110 enable sharing of measurements to perform a discharge date determination. In some examples, discharge date determination is made based on collection of data from multiple devices within the patient environment 104 including the mobility detection devices 112, the patient support apparatus 116, as well as information from outside the patient environment 104, such as the EMR 128 and/or third party server(s) 134.

Walk assist devices 118 comprise devices that the patient 106 uses to help maintain balance and stability as they walk or otherwise move around the patient environment 104. Examples of walk assist devices 118 include, without limitation, a walker, a walking cane, a bathroom transportation aide, a patient lift, and/or any other suitable device.

Wearable devices 120 comprise one or more motion sensors attached to the body of the patient 106 to detect the mobility of the patient 106. In some instances, the one or more motion sensors are attached directly to the body of the patient 106 such as in a body patch. In other examples, the one or more motion sensors are embedded in clothing worn by the patient 106, such as socks or in an accessory worn by the patient 106, such as a wristwatch, a bracelet, an ankle band, or a chest band. In some examples, wearable devices 120 include GPS sensors, accelerometers, wireless real-time locating system (RTLS) tags, and/or any other suitable devices to track the mobility of the patient 106. In some examples, a GPS sensor is helpful to track the location of a patient diagnosed with Alzheimer's. Wearable devices 120 transmit movement data detected from the patient 106 to the controller 108 of the patient support apparatus 116. In some examples, the wearable devices 120 utilize Wi-Fi, Zigbee, and other wireless technologies and Internet of Things (IOT) communication standards to send the movement data to the controller 108.

In some examples, wearable devices 120 are part of a real-time locating system (RTLS) that is used to detect and track movements of the patient 106. In some examples, the wearable devices 120, when used together with the patient support apparatus 116, walk assist devices 118, and mounted devices 122, increase the spatial resolution of the RTLS.

Mounted devices 122 comprise devices that are mounted to a wall, a ceiling, or to another device within the patient environment 104, such as the patient support apparatus 116, a digital whiteboard, a portable stand, a vital sign monitor, a chair, and the like. In some examples, mounted devices 122 include one or more video cameras or radar transceivers that can be used to detect and track the movements of the patient 106 within the patient environment 104.

As illustrated, patient environment 104 further includes camera(s) 124. Camera(s) 124 are configured to capture images of a sub-environment within the environment 100. For example, the camera(s) 124 may be configured to capture images of a room, a curtained area, a hallway, an operating room, or another type of sub-environment within the clinical setting. The clinical setting, for example, may be a hospital, medical clinic, hospice, or any other type of managed care facility. In some examples, the camera(s) 124 comprise one or more thermal camera(s), three-dimensional camera(s), infrared camera(s), or any other suitable camera.

The images captured by the camera(s) 124 may include one or more objects. As used herein, the term “object,” and its equivalents, may refer to a portion of an image that can be interpreted as a single unit and which depicts a single subject depicted in the image. Examples of subjects include machines, devices, equipment, as well as individuals depicted in the images. In some implementations, an object is a portion of a subject that is depicted in the images. For example, a head, a trunk, or a limb of an individual depicted in the images can be an object.

As illustrated in FIG. 1, the controller 108 and/or controller 110 communicates with mobile device(s) 126a-126c (referred to collectively as “mobile device(s) 126”) via the network(s) 114. For instance, the controller 108 and/or controller 110 may communicate changes in the mobility status of the patient 106 directly to mobile device(s) 126 (e.g., computing device(s), cell phone, tablet, laptop, beeper, etc.). In some examples, a mobile device 126 is associated with a caregiver in the healthcare facility, a family member of the patient 106, a doctor, a nurse, a social worker, etc. In some examples, the discharge management system 102 provides an instance of application to each mobile device 126, where the instance of the application is configured to enable each caregiver, doctor, nurse, social worker, and/or family member to monitor alerts and/or notifications regarding the mobility status of the patient 106, and to conduct voice and video communications with the patient 106, via the mobile device 126. In some examples, the controller 108 and/or controller 110 may disable alerts based on detecting that the patient 106 is sleeping.

In some examples, the controller 108 and/or controller 110 communicates changes in the mobility status of the patient 106 to a nurse call server 130 via the network(s) 114. In some examples, the nurse call server 130 is configured to manage communications sent between mobile devices 126, as well as communications between the mobile devices 126 and the nurses' station 160.

In some examples, the controller 108 and/or controller 110 communicates changes in the mobility status of the patient 106 to a nurses' station 132 via the network(s) 114. The nurses' station 132 comprises an area in a healthcare facility, such as a hospital and/or nursing home, where caregivers such as nurses' and/or other staff work when not working directly with the patient 106 (e.g., such as where administrative tasks are performed). The nurses' station 132 includes one or more computing devices connected to the network(s) 114.

In some examples, the controller 108 and/or controller 110 communicates with and/or accesses health data from one or more third party server(s) 134. For instance, in some examples, the third-party server(s) 134 comprise servers associated with a healthcare facility (e.g., such as a separate hospital, a doctor's office, etc.). In this example, the controller 108 and/or controller 110 accesses patient health records from the third-party server(s) 134, such as at intake of the patient 106.

In some examples, the controller 108 and/or controller 110 receives input indicating intake information associated with a patient 106. For instance, when the patient 106 is initially admitted, the controller 108 and/or controller 110 receives information associated with the patient 106 (e.g., demographic information, injury information, health information etc.). In some examples, the controller 108 and/or controller 110 determines, based at least in part on the information, a target discharge date (e.g., when the patient is expected to be discharged), mobility requirements (e.g., goal mobility for the patient) for discharge, and/or discharge event(s) (e.g., what event(s) (e.g., procedures, appointments, paperwork, etc., need to occur for the patient to be discharged) for the patient. In some examples, the target discharge date is input by a caregiver. In other examples, the target discharge date is determined by the controller 108 and/or controller 110, based at least in part on the reason for intake (e.g., injury, procedure, etc.), an average recovery time associated with the reason for intake, age of the patient, health of the patient, scheduling information associated with the healthcare facility, and/or any other information to which the controller 108 and/or controller 110 has access. For instance, the mobility requirements are determined based on mobility of the patient at intake (e.g., how mobile the patient was when admitted to the healthcare facility). In some examples, the mobility requirements are based on a location to which the patient will be discharged to (e.g., assisted living facility, independent living facility, home of the patient, etc.).

In some examples, the controller 108 and/or controller 110 receives a mobility status of the patient 106 that is based on movement data received from a plurality of sensors within the patient environment 104. For instance, the mobility status is calculated or scaled to represent as a mobility score such as a bedside mobility assessment tool (BMAT) score, an activity measure for post-acute care (AM-PAC) score, or a proprietary mobility score. The mobility score can be based on at least the movement data and patient demographics and physiological data acquired from an electronic medical record (EMR) 128 (alternatively termed electronic health record (EHR)) of the patient 106. The EMR 128 is stored in one or more servers. As noted above, the controller 108 and/or controller 110 may utilize the movement data and additional parameters of the patient 108 to determine a variety of clinical assessments and scores (e.g., a Braden's mobility score, sepsis prediction score, pressure injury risk score, falls risk score, etc.). In some examples, the controller 108 and/or controller 110 may determine, based on the additional parameters and/or movement data that one or more measurements taken by sensor(s) (e.g., such as mobility detection device(s) 112 or any other sensor) is inaccurate. For instance, the controller 108 and/or controller 110 may determine that the patient's heart rate has not increased in response to sensing movement by the patient. In this example, the controller 108 and/or controller 110 may generate and transmit an alert to a caregiver of the patient, where the alert indicates that one or more of the sensor(s) may be inaccurate.

In some examples, controller 108 and/or controller 110 sends the aggregated movement data to an external server (e.g., such as nurse call server 130, third part server(s) 134, EMR 128, etc.) connected to the network(s) 114. Upon receipt of the aggregated movement data, one or more algorithms stored on a memory of the external server, when executed by a processor of the external server, cause the external server to process the aggregated movement data to determine the mobility status of the patient 106. The external server can be an on-premises or edge-server located within the healthcare facility, or can be a cloud-based server.

The controller 108 and/or controller 110 processes the aggregated movement data collected from the patient support apparatus 116 and/or mobility detection devices 112 to determine the mobility status of the patient 106 within the patient environment 104. For instance, the controller 108 and/or controller 110 may access one or more algorithms stored on a memory of the controller 108 and/or controller 110. The controller 108 and/or controller 110 then transfers the mobility status of the patient 106 to the network(s) 114.

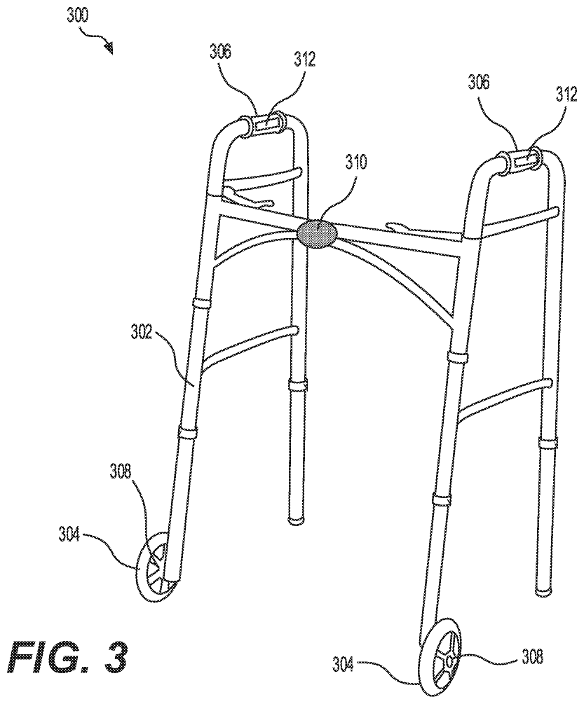

In some examples, the controller 108 and/or controller 110 automatically documents patient 106 mobility including transitions from the patient support apparatus 116 to a chair within the patient environment 104, from the patient support apparatus 116 to the walker 300 (described below with regard to FIG. 3) to a chair in the patient environment 104, time spent outside the patient support apparatus 116, time spent in the chair, time spent outside of the patient environment 104, number of steps per day, maximum length of walk and daily walking trends, and the like. The controller 108 can distinguish between unassisted motions and caregiver-assisted motions through sensor readings on the patient support apparatus 116 and mobility detection devices 112, and other devices within the patient environment 104 including RTLS devices that can determine patient to caregiver proximity.

The controller 108 and/or controller 110 determines an estimated discharge date and/or a confidence metric associated with the estimated discharge date of the patient. In some examples, the estimated discharge date is based at least in part on the mobility information, EMR, etc. For instance, the estimated discharge date may be based at least in part on identifying, by the controller 108 and/or controller 110 that each of the discharge event(s) required for discharge have occurred on schedule. Additionally, or alternatively, the estimated discharge date may be determined based on identifying that the mobility of the patient is progressing at a particular speed relative to an average mobility progression (e.g., based on intake information, age of patient, health, intake reason, procedure(s), etc.). The controller 108 and/or controller 110 also determines a confidence metric associated with the estimated discharge date, which indicates a likelihood that the patient will be discharged on the estimated discharge date.

For instance, the controller 108 and/or controller 110 compares a current mobility status of the patient 106 against a prior mobility assessment of the patient 106 stored in the EMR 128. The comparison determines whether the current mobility status of the patient 106 matches the prior mobility assessment stored in the EMR 128. An inconsistency in the EMR 128 may result from a recent change in the mobility status of the patient 106 that has not yet been updated in the EMR 128 and/or may result from the prior mobility assessment stored in the EMR 128 being incorrect. When the controller 108 and/or controller 110 determines that an inconsistency exists, the controller 108 can generate an alert that can be sent to the caregivers via the network(s) 114. The controller 108 can provide the caregivers with the updated mobility status of the patient 106. In some examples, the controller 108 automatically updates the mobility assessment of the patient 106 stored in the EMR 128. Alternatively, the controller 108 can prompt a caregiver to validate the updated mobility assessment of the patient 106 before storing the updated mobility assessment in the EMR 128.

The controller 108 and/or controller 110 determines whether the estimated discharge date and/or confidence metric is greater than one or more threshold(s). For instance, the controller 108 and/or controller 110 may determine whether the estimated discharge date is (i) earlier than the target discharge date by more than a threshold period of time, (ii) later than the target discharge date by more than a threshold period of time, and/or (iii) whether the mobility requirement(s) and/or discharge event(s) are achievable by the target discharge date. In some examples, the controller 108 and/or controller 110 determines whether the confidence metric is greater than or equal to one or more threshold(s) (e.g., 70%, 80%, 90%, or any other suitable threshold). Where the estimated discharge date and the confidence metric meet (e.g., is greater than or equal to) the one or more threshold(s), the controller 108 and/or controller 110 may take one or more action(s). In some examples, the action(s) comprise generating an alert to caregivers, generating a recommendation for the patient, scheduling an appointment and/or procedure for the patient, changing the target discharge date, updating the EMR 128, etc.).

For instance, when the patient 106 has a prior mobility assessment indicating that the patient 106 is mobile, and the controller 108 and/or controller 110 detects from the aggregated movement data that the patient 106 has not left the patient support apparatus 116 after a predetermined period of time (e.g., 24 hours), the controller 108 and/or controller 110 can send an alert to a caregiver or family member. When the controller 108 and/or controller 110 detects that the mobility of the patient 106 has declined or is trending in a manner at odds with the patient 106 discharge plan, the controller 108 can send an alert to the caregiver or family member. The controller 108 and/or controller 110 can order physical therapy, or provide a recommendation for physical therapy, when a decline in mobility for the patient 106 is detected. Additionally, or alternatively, the controller 108 and/or controller 110 may recommend changing the target discharge date and/or a discharge location (e.g., a location where the patient 108 will be discharged to, such as a home, care facility, rehabilitation facility, etc.).

In some examples, the controller 108 and/or controller 110 recommends ordering a walk assist device such as a walker and/or walking cane for the patient 106, or suggest an alternative care pathway based on the mobility of the patient 106. Additionally, the controller 108 can generate an advanced mobility report on timing of patient turns, frequency of patient turns, whether the patient turns are assisted or not, time spent in each position, and body and extremity (major and minor) movement statistics.

In the perioperative context, the controller 108 and/or controller 110 alerts a caregiver and/or staff of the healthcare facility that the patient 106 needs to be moved before surgery while in a PreOp area. The position of the patient 106 and cumulative time on a surface such as the patient support apparatus 116 across the PreOp, intra-op, and PostOp areas can be communicated by the controller 108 and/or 110 via the network(s) 114 to the next level of care within a surgical unit or after discharge from PostOp, as well as to nursing managers to provide suggestions on adjusting the support angle and mattress firmness of the patient support apparatus 116, as well as other surface changes, next best caregiver action, and the like.

Accordingly, various methods described herein may include monitoring mobility of a patient and predict an estimated discharge date and/or confidence metric in real-time based on real-time sensor data, aggregated data, health information, EMR, etc., to provide a more accurate and personalized determination of patient discharge date, thereby avoiding the day of discharge surprise. The methods also enable a healthcare facility to take action(s) to help the patient meet a particular discharge date and/or keep a patient and/or families informed, thereby avoiding the day of discharge surprise. This enables better care for patients and may reduce the length of stay in a healthcare facility with little to no additional costs to the patient and/or healthcare facility.

FIG. 2 illustrates an example of the patient support apparatus 116 associated with the patient environment 104 of FIG. 1. While FIG. 2 depicts the patient support apparatus 116 as comprising a hospital bed, alternative examples are possible where the patient support apparatus 116 comprises a chair, a recliner, surgical table, and/or any other suitable type of support apparatus. Accordingly, the description provided herein is not limited to hospital beds.

As illustrated in FIG. 2, patient support apparatus 116 comprises a frame 200 that supports a mattress 202. The mattress 202 is flexible and conforms to the profile of the frame 200 as the orientation of the frame 200 is adjusted between horizontal and upright orientations. The mattress 202 includes one or more bladders (not shown) that adjust the firmness of the mattress 202 as they are be inflated and deflated.

Patient support apparatus 116 further includes sensors 204a-204f (referred to collectively as “sensors 204”). Sensors 204 are configured to detect movements of the patient 106 (e.g., such as while the patient 106 rests on and/or is in contact with the patient support apparatus 116). In some examples, one or more sensors 204 include load cells, such as piezoelectric sensors, that produce a voltage or current signal indicative of a weight impressed on the load cell from the body of the patient 106. In some examples, one or more sensors 204 include pressure sensors that measure a resistance inversely proportional to the pressure applied on the sensors from the weight of the body of the patient 106. Additionally, or alternatively, sensors 204 include a variety of other types of sensors, including capacitance sensors, to detect the movement of the patient 106 while the patient 106 rests on the patient support apparatus 116. As illustrated in FIG. 2, sensors 204 are mounted to the frame 200 and are positioned under the mattress 206. In some examples, the sensors 204 are coupled to a top or bottom surface of the mattress 202, and/or positioned within an interior region of the mattress 202.

The frame 200 further includes a left siderail assembly having at least one left siderail mounted on the left side of the frame and a right siderail assembly having at least one right siderail mounted on the right side of the frame. As illustrated in FIG. 2, the left siderail assembly includes an upper left siderail 210 and a lower left siderail 212, and the right siderail assembly includes an upper right siderail 214 and a lower right siderail 216. Each siderail 210-216 is positionable at a deployed position at which its upper edge is higher than the top of the mattress 202 and at a stowed position at which its upper edge is lower than the top of the mattress 202. When configured in the deployed position, a siderail (e.g., 210-216) prevents the patient 106 from exiting the patient support apparatus 116. When configured in the stowed position, a siderail allows the patient 106 to enter and exit the patient support apparatus 116. In the example embodiment illustrated in FIG. 2, the upper left siderail 210, lower left siderail 212, and upper right siderail 214 are in the deployed position, and the lower right siderail 216 is in the stowed position.

Patient support apparatus 116 further includes wheels 218 to facilitate the portability of the patient support apparatus 116, and a headboard 220 and a footboard 222. In certain embodiments, the footboard 222 is removable from the foot end of the frame 200 in order to accommodate occupant egress from the foot end. For example, in certain embodiments, the patient support apparatus 116 can be adjusted so that its profile mimics that of a chair.

Patient support apparatus 116 further includes a user interface 230. In some examples, user interface 230 comprises one or more controls that are used to control and adjust the various functions of the patient support apparatus 116. For example, the user interface 230 includes input devices such as buttons, switches, and/or a touchscreen display to adjust the position of the frame 200, the firmness of the mattress 202, reset one or more alarms, and control other functions of the patient support apparatus 116. In the example shown in FIG. 2, the user interface 230 is positioned on the upper right siderail 214.

Patient support apparatus 116 further includes audio assembly 240 that has at least one speaker to provide audio instructions to the patient 106. For example, the audio assembly 240 and speaker can be used to provide audio instructions for the patient 106 to self-turn. In some examples, the audio assembly 240 includes a microphone that is configured to enable two-way audio communication between the patient 106 and caregivers.

In some examples, one or more of the siderails 210-216 are provided with patient strength sensors 242. In some examples, instructions are provided through the audio assembly 240 for the patient 106 to apply pressure and/or force to (e.g., to squeeze) the patient strength sensors 242 for assessing patient 106 strength remotely. The controller 108 receives strength data from the patient strength sensors 242. In some examples, the controller 108 combines the strength data with the aggregated movement data to determine the strength and mobility of the patient 106. The controller 108 transmits the strength data to the EMR 128 via the network(s) 114. In some examples, the controller 108 transmits the strength data to the mobile devices 126 and/or nurses' station 132 for display.

Accordingly, in this way, the controller 108 and/or controller 110 may monitor mobility of a patient and predict an estimated discharge date and/or confidence metric in real-time based on real-time sensor data, aggregated data, health information, EMR, etc., to provide a more accurate and personalized determination of patient discharge date, thereby avoiding the day of discharge surprise.

FIG. 3 illustrates a walker 300 that is an example of a walk assist device shown in FIG. 1. The walker 300 can be used in a healthcare facility or in the home of the patient 106 home to transmit movement data and alerts to the controller 108 and/or controller 110. In some examples, the walker 300 utilizes Wi-Fi, Zigbee, cellular networks, and/or other wireless technologies and/or Internet of Things (JOT) communication standards to send the movement data and alerts to the controller 108 and/or controller 110. The controller 108 and/or controller 110 can forward the alerts to the EMR 128, nurse call server 130, nurses' station 132, mobile devices 126, etc. via the network(s) 114.

The walker 300 comprises a lightweight frame 302 with one or more wheels 304 attached the frame. The walker 300 further includes handles 306 that are gripped by the patient 106 when using the walker 300 to maintain their balance and stability while walking.

The walker 300 includes one or more sensors 308 connected to the wheels 304 to detect rotation of the wheels 304 to measure the frequency of use, gait, speed, acceleration, number of steps, equilibrium, wait times between movements, falls or near falls, pressure and/or weight supported by the walker 300, and other measurements of the patient 106. Alternatively, or in addition to the sensors 308, the walker 300 may also have additional sensors 310 such as a GPS sensor, accelerometer, wireless real-time locating system (RTLS) tag, and similar sensors to track the mobility of the patient 106. For instance, a GPS sensor may be helpful tracking the location of a patient diagnosed with Alzheimer's, such as when the patient leaves the patient environment 104 without assistance from a caregiver or family member.

Walker 300 further includes one or more sensors 312 connected to the handles 306 that measure additional parameters such as the equilibrium or balance of the patient 106 and one or more physiological variables of the patient 106 while using the walker 300. Examples of the physiological variables that can be measured from the sensors 312 include heart rate, pulse, and SpO₂. The sensors 312 can also measure the patient 106 strength when gripping the handles 306 to detect whether there is a change in grip showing patient fatigue, weakness, or deterioration.

In certain examples, the walker 300 comprises a controller (not shown) that is configured to trigger an alert containing an alert type and a device location when the patient 106 experiences a lack of equilibrium, a fall, a pause between movements that exceeds a threshold, or leaves a designated area such as the patient environment 104 without assistance from a caregiver. The controller (not shown) of the walker 300 wirelessly sends the alert to the controller 108 and/or controller 110 of the patient support apparatus 116, and the controller 108 can forward the alert to the network(s) 114. The controller 108 and/or controller 110 can then transmit the alert to at least one of the mobile devices 126, EMR server 140, nurse call server 150, and/or nurses' station 160.

In some examples, the walker 300 includes a self-charging capability, such as from transforming the kinetic energy from the rolling of the wheels 304 into electrical energy to charge the onboard electronics. Additionally, the walker 300 can include charger plugs or be configured for in-place charging when parked on top of a charging base to charge the onboard electronics when the walker 300 is not in use. Typical daily operation of the walker 300 can be sufficient for the walker 300 to operate without requiring charging from the charger plugs or in-place charging. However, the walker 300 can remain charged for a predetermined period of time (e.g., one week) while remaining idle. In some examples, the walker 300 operates like a regular walker when uncharged.

In some examples, such as where walker 300 is used in the home of the patient 106, transmits movement data to an external application (not shown) for tracking the patient 106 movements while in the home. In some examples, the external application is used by the physician of the patient 106 to monitor the progress and/or recovery of the patient 106, and to compare movement trends between the healthcare facility where the patient 106 was admitted and home of the patient 106, and vice versa.

Accordingly, various methods described herein may include monitoring mobility of a patient and predicting an estimated discharge date and/or confidence metric in real-time based on real-time sensor data, aggregated data, health information, EMR, etc., to provide a more accurate and personalized determination of patient discharge date, thereby avoiding the day of discharge surprise. The methods may also include enabling a healthcare facility to take action(s) to help the patient meet a particular discharge date and/or keep a patient and/or families informed, thereby avoiding the day of discharge surprise. This enables better care for patients and may reduce the length of stay in a healthcare facility with little to no additional costs to the patient and/or healthcare facility.

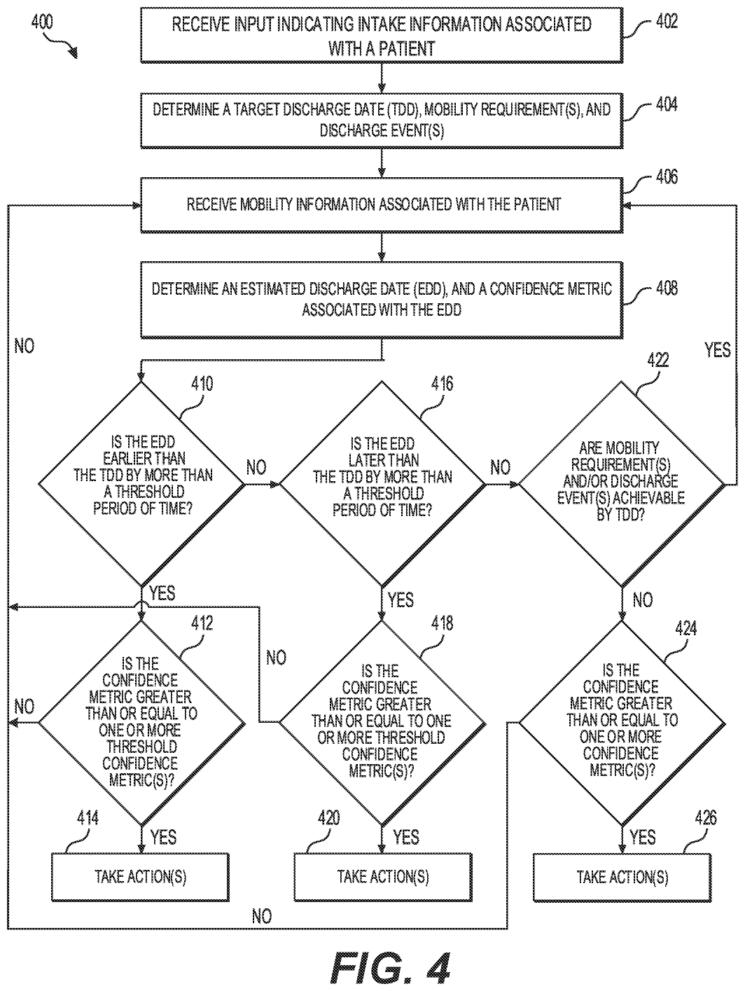

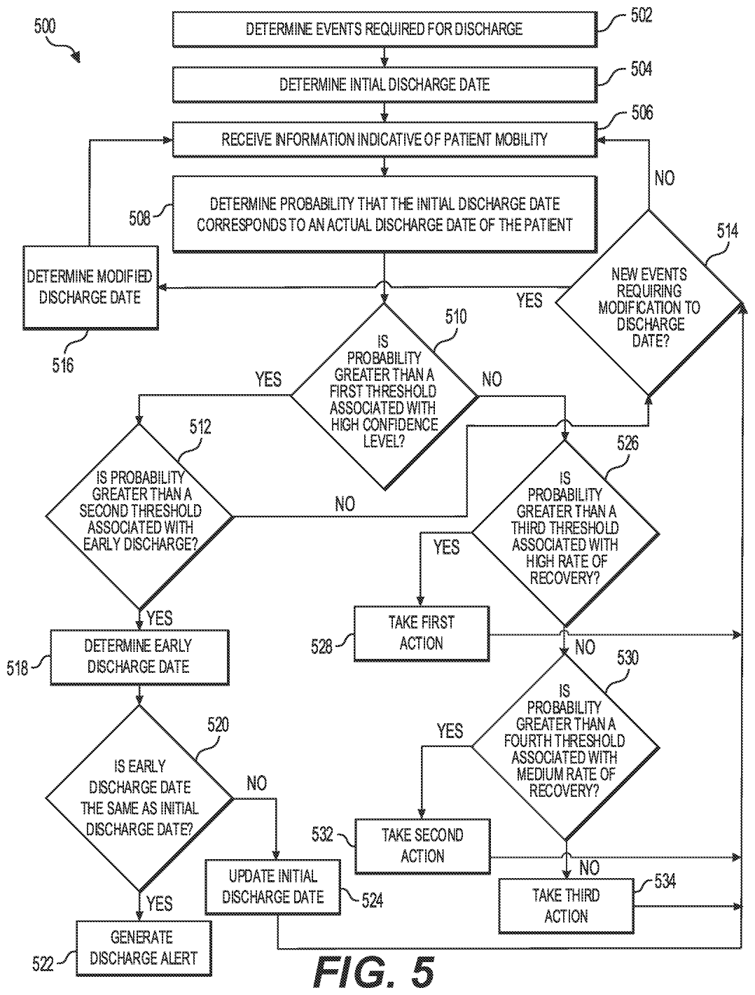

FIGS. 4 and 5 illustrate example methods 400, 500 associated with the discharge monitoring system shown in FIGS. 1-3. The example methods 400, 500 of FIGS. 4 and 5, respectively, are illustrated as logical flow graphs, each operation of which represents a sequence of operations that may be implemented in hardware, software, or a combination thereof. In the context of software, the operations represent computer-executable instructions stored on one or more computer-readable storage media that, when executed by one or more processors, perform the recited operations. Generally, computer-executable instructions include routines, programs, objects, components, data structures, and the like that perform particular functions or implement particular abstract data types. The order in which the operations are described is not intended to be construed as a limitation, and any number of the described operations may be combined in any order and/or in parallel to implement the processes. Although any of the processes or other features described with respect to the methods 400 and/or 500 may be performed by processor(s) and/or controller(s) 108 and/or 110, for ease of description, the example methods 400 and 500 will be described below as being performed by the controller 108 of the discharge management system 102 unless otherwise noted.

As illustrated in FIG. 4, at 402, the controller 108 receives input indicating intake information associated with a patient 106. In some examples, the input is received from a caregiver (e.g., manual input) from a mobile device 126 and/or nurses' station 132. In some examples, the input is received from the EMR 128 and/or third-party server(s) 134.

At 404, the controller 108 determines a target discharge date (TDD), mobility requirement(s), and discharge event(s). In some examples, initial discharge event(s) are manually input by a caregiver. In some examples, discharge event(s) and/or mobility requirement(s) are determined automatically—e.g., based on the intake input. For instance, the controller 108 may determine the event(s) required for discharge is based on a type of condition and/or treatment associated with the patient. In some examples, the discharge event(s) are identified by the controller 108 as a predetermined set stored in memory of the controller and/or system that is accessed by the controller 108 in response to input indicating the type of condition and/or treatment. As noted above, the mobility requirement(s) may be determined based on a location to which the patient is being released (e.g., home, independent living facility, assisted living facility, skilled nursing facility, rehabilitation facility, etc.). In this example, the controller identifies the discharge location as the patient's home. For instance, the controller may access the EMR of the patient and determine, based on information taken at intake, a location to which the patient will be discharged to. The controller 108 may identify mobility requirement(s) based on characteristics associated with the patient's home, such as being able to walk up and down stairs, etc. In some examples, the controller 108 outputs one or more of the TDD, the mobility requirement(s), and/or the discharge event(s) to a display associated with the caregiver. In some examples, the display is associated with the nurses' station and/or mobile devices 126 described in FIG. 1. Accordingly, the controller 108 may receive input indicating one or more of confirmation (e.g., approval), change (e.g., updating), and/or removal of one or more of the TDD, mobility requirement(s), and/or discharge event(s).

At 406, the controller 108 receives mobility information associated with the patient. As described above, the mobility information may comprise information from one or more of (i) the plurality of sensors within the patient environment 104, the EMR, third-party server(s), etc.

At 408, the controller 108 determines an estimated discharge date (EDD) and a confidence metric associated with the EDD. In some examples, the EDD is determined based on an events progression speed and/or a mobility progression speed. In some examples, one or more algorithms, data plots, graphs, lookup tables including empirical data, neural networks, and/or other items may be utilized by the controller 108 to determine the EDD. In some examples, the EDD is determined based on one or more formulas, including a regression formula, decision tree, random forest, and/or gradient boosting machine. For instance, the EDD may be based on a mobility progression speed, which is determined using one or more algorithms, such as a regression formula:

rate of change=∝*age+β*gender+χ*BMI

where ∝, β, and χ represent coefficient constants used to weight various factors associated with the patient. For instance, one or more of ∝, β, and/or χ may represent demographic factors associated with the patient (e.g., such as age, gender, BMI, potential comorbidities of the patient), preadmission mobility level, and/or any other suitable characteristic associated with the patient. In some examples, the controller 108 and/or controller 110 determines the EDD using the regression formula. For instance, the controller 108 and/or 110 may compare a first mobility level associated with the mobility level required to be discharged with a current mobility level of the patient. Based on a difference between the two mobility levels the controller 108 and/or controller 110 may determine an amount of time required for the patient to improve, which may be based on any of the factors described herein (e.g., demographic factors and/or any other characteristics). Accordingly, the EDD may be determined based on a current rate of change and the amount of time required for the patient to improve. In some examples, a confidence metric associated with the mobility progression speed is determined. For instance, the controller may determine the confidence metric for mobility progression speed based on a percentage of the time estimate used in the regression formula (e.g., such as +/−10%, 20%, 30%, or the like).

In some examples, the events progression speed is determined using one or more algorithms, such as a Monte Carlo analysis. For instance, the Monte Carlo analysis outputs a distribution of EDD estimates that indicate a likelihood that the patient will be discharged by the TDD. In this example, the Monte Carlo analysis outputs a histogram, where the X-axis corresponds to time with gradations based on a particular interval (e.g., hours, days, etc.). The Y-axis corresponds to a number of EDD estimates that fall within each gradation. In some examples, the controller determines the confidence metric based on comparing the distribution of EDD with the TDD. For instance and as described in greater detail below with regard to FIGS. 4 and 5, where a percentage of the EDD estimates exceed the TDD (e.g., is later than the TDD and/or earlier than the TDD by more than a threshold amount), the controller may determine that an action is required. In some examples, the confidence metric is determined based on a width of the distribution of EDD estimates output by the Monte Carlo analysis and/or a percentage of the EDD estimates that fall outside of the distribution of EDD estimates.

In some examples, the events progression speed is determined based on one or more anticipated inputs. For instance, anticipated inputs include an estimated length of an event, an estimated variation of event length, an estimated uncertainty of the event length, and/or whether one or more event(s) are run in a particular sequence (e.g., such as whether the patient has met required physical condition(s) by the time the event start(s)). For instance, a discharge event may comprise a procedure. In this example, if the patient is estimated to meet the pre-procedure physical conditions (e.g., such as blood pressure, heart rate, etc.), then the controller may determine that the event(s) (e.g., pre-op and the procedure) can run in a sequence.

In some examples, the controller 108 receives one or more of the inputs from a caregiver. In some examples, one or more of the inputs are determined by the controller using machine learning mechanisms and using information associated with a plurality of patients, procedures, etc. Such machine-learning mechanisms include, but are not limited to supervised learning algorithms (e.g., artificial neural networks, Bayesian statistics, support vector machines, decision trees, classifiers, k-nearest neighbor, etc.), unsupervised learning algorithms (e.g., artificial neural networks, association rule learning, hierarchical clustering, cluster analysis, etc.), semi-supervised learning algorithms, deep learning algorithms, etc.), statistical models, etc. In at least one example, machine-trained data models can be stored in memory associated with the controller 108.

At 410, the controller 108 determines whether the EDD is earlier than the TDD by more than a threshold period of time. In some examples, the threshold period of time comprises 6 hours, 8 hours, 12 hours, 24 hours, and/or any other suitable time period. As noted above, in some examples, the threshold period of time is pre-set by a caregiver of the patient 106. In some examples, the threshold period of time is adjustable by the caregiver (e.g., such that caregiver can change the threshold period of time).

Where the controller 108 determines that the EDD is earlier than the TDD by more than the threshold period of time (Step: 410—Yes), the method 400 continues to 412. At 412, the controller determines whether the confidence metric is greater than or equal to a threshold confidence metric. For instance, as described above, the one or more threshold confidence metrics (e.g., 70%, 80%, 90%, or any suitable threshold) may be pre-set by a caregiver (e.g., a nurse, a doctor, etc.) at intake of the patient. In some examples, the one or more threshold confidence metrics may be changed and/or updated by a caregiver while the patient is in the healthcare facility.

Where the controller 108 determines that the confidence metric is greater than or equal to a threshold confidence metric (Step: 412—Yes), the method continues to 414. At 414, the controller 108 takes one or more action(s). In some examples, the action(s) taken at 414 are based at least in part on one or more of the thresholds (e.g., the threshold period of time and/or the threshold confidence metric). For instance, in some examples the threshold(s) are pre-set by a caregiver at the hospital. For instance, a nurse may pre-set the confidence metric threshold to be 70% and/or the threshold period of time to be 12 hours. Accordingly, where the controller determines that one or both threshold(s) are met, the controller generates an alert to notify the nurse. In some examples, the controller 108 sends the alert to the mobile device 126 and/or nurses' station 132 for display. In some examples, the alert comprises a recommendation to move the TDD to an earlier date and/or an option to accept or decline the recommendation. In this example, the controller 108 receives from the mobile device 126 and/or nurses' station 132 input indicating the nurses' selection of accepting the recommended change to the TDD or declining the recommended selection. Where the input indicates acceptance of the change to the TDD, the controller 108 sends, to the EMR 128, a message to cause the EMR 128 to update the TDD associated with the patient 106. In some examples, the controller 108 automatically sends the message to the EMR to update the TDD in response to the pre-set threshold(s) being met and/or exceeded (e.g., without input from the caregiver). In some examples, the alert comprises an indication(s) associated with one or both threshold(s). For instance, where the alert comprises a recommendation to change the TDD to a later date, the alert may indicate one or more factor(s) associated with the recommendation. In this example, the alert may indicate that the factor(s) (e.g., such as factor(s) associated with determining mobility progression speed and/or event progression speed described above).

Where the controller 108 determines that the EDD is not earlier than the TDD by more than the threshold period of time (Step: 410—No), the method proceeds to 416. At 416, the controller 108 determines whether the EDD is later than the TDD by more than a threshold period of time. In some examples, the threshold period of time comprises 6 hours, 8 hours, 12 hours, 24 hours, and/or any other suitable time period. As noted above, in some examples, the threshold period of time is pre-set by a caregiver of the patient 106. In some examples, the threshold period of time is adjustable by the caregiver (e.g., such that caregiver can change the threshold period of time).

Where the controller 108 determines that the EDD is later than the TDD by more than the threshold period of time (Step: 416—Yes), the method continues to 418. At 418, the controller 108 determines whether the confidence metric is greater than or equal to one or more threshold confidence metrics. For instance, as described above, the one or more threshold confidence metrics (e.g., 70%, 80%, 90%, or any suitable threshold) may be pre-set by a caregiver (e.g., a nurse, a doctor, etc.) at intake of the patient. In some examples, the one or more threshold confidence metrics may be changed and/or updated by a caregiver while the patient is in the healthcare facility.

At 420, the controller 108 takes one or more action(s). In some examples, the action(s) taken at 420 are based at least in part on one or more of the thresholds (e.g., the threshold period of time and/or the threshold confidence metric). For instance, in some examples the threshold(s) are pre-set by a caregiver at the hospital. For instance, a nurse may pre-set the confidence metric threshold to be 70% and/or the threshold period of time to be 12 hours. Accordingly, where the controller determines that one or both threshold(s) are met, the controller generates an alert to notify the nurse. In some examples, the controller sends the alert to the mobile device 126 and/or nurses' station 132 for display. In some examples, the alert comprises a recommendation to move the TDD to an earlier date and/or an option to accept or decline the recommendation. In this example, the controller receives from the mobile device 126 and/or nurses' station 132 input indicating the nurses' selection of accepting the recommended change to the TDD or declining the recommended selection. Where the input indicates acceptance of the change to the TDD, the controller sends, to the EMR 128, a message to cause the EMR 128 to update the TDD associated with the patient 106. In some examples, the controller automatically sends the message to the EMR 128 to update the TDD in response to the pre-set threshold(s) being met and/or exceeded (e.g., without input from the caregiver). In some examples, the alert comprises an indication(s) associated with one or both threshold(s). For instance, where the alert comprises a recommendation to change the TDD to an earlier date, the alert may indicate one or more factor(s) associated with the recommendation. In this example, the alert may indicate that the factor(s) (e.g., such as factor(s) associated with determining mobility progression speed and/or event progression speed described above).

Where the controller 108 determines that the EDD is not later than the TDD by more than the threshold period of time (Step: 416—No), the method proceeds to 422. At 422, the controller 108 determines whether the mobility requirement(s) and/or discharge event(s) achievable by the TDD. In some examples, the controller 108 determines whether one or more of the mobility requirement(s) and/or discharge event(s) are achievable by the TDD using the events progression speed and/or mobility progression speed. For instance, the controller 108 may access previous determination(s) of mobility progression speed and/or event progression speed. The controller determines, based on the mobility progression speed and/or event progression speed determined at 408, what the anticipated mobility of the patient will be on the TDD and/or whether one or more discharge event(s) will not have occurred by the TDD.

Where the controller 108 determines that the mobility event(s) and/or discharge event(s) are achievable by the TDD (Step: 422—Yes), the method returns to 406 and the controller continues to monitor information associated with the patient.

Where the controller 108 determines that one or more mobility event(s) and/or discharge event(s) are not achievable by the TDD (Step: 422—No), the method proceeds to 424. At 424, the controller determines whether the confidence metric is greater than one or more confidence threshold(s). As described above, the one or more threshold confidence metrics (e.g., 70%, 80%, 90%, or any suitable threshold) may be pre-set by a caregiver (e.g., a nurse, a doctor, etc.) at intake of the patient. In some examples, the one or more threshold confidence metrics may be changed and/or updated by a caregiver while the patient is in the healthcare facility.

Where the controller 108 determines that the confidence metric is not greater than the one or more confidence threshold(s) (Step: 424—No), the method returns to 406 and the controller continues to receive and monitor information associated with the patient.

Where the controller 108 determines that the confidence metric is greater than one or more confidence threshold(s) (Step: 424—Yes), the method proceeds to 426. At 426, the controller 108 takes one or more action(s). In some examples, the action(s) taken at 426 are based at least in part on one or more of the thresholds (e.g., the threshold period of time and/or the threshold confidence metric). For instance, in some examples the threshold(s) are pre-set by a caregiver at the hospital. For instance, a nurse may pre-set the confidence metric threshold to be 70% and/or the threshold period of time to be 12 hours. Accordingly, where the controller determines that one or both threshold(s) are met, the controller generates an alert to notify the nurse. In some examples, the controller sends the alert to the mobile device 126 and/or nurses' station 132 for display. In some examples, the alert comprises a recommendation to move the TDD to an earlier date and/or an option to accept or decline the recommendation. In this example, the controller receives from the mobile device 126 and/or nurses' station 132 input indicating the nurses' selection of accepting the recommended change to the TDD or declining the recommended selection. Where the input indicates acceptance of the change to the TDD, the controller 108 sends, to the EMR 128, a message to cause the EMR 128 to update the TDD associated with the patient 106. In some examples, the controller 108 automatically sends the message to the EMR 128 to update the TDD in response to the pre-set threshold(s) being met and/or exceeded (e.g., without input from the caregiver). In some examples, the alert comprises an indication(s) associated with one or both threshold(s). For instance, where the alert comprises a recommendation to change the TDD to an earlier date, the alert may indicate one or more factor(s) associated with the recommendation. In this example, the alert may indicate that the factor(s) (e.g., such as factor(s) associated with determining mobility progression speed and/or event progression speed described above).

FIG. 5 illustrates another example method 500 associated with the example discharge management system. As illustrated in FIG. 5, at 502, the controller 108 determines events required for discharge. As described above, the events required for discharge comprise any event associated with scheduling, procedure(s), treatment(s), paperwork, follow-up(s), etc. that may be required to discharge the patient from the healthcare facility. Additionally, or alternatively, the events may comprise mobility requirement(s) as described above.

At 504, the controller 108 determines an initial discharge date. In some examples, initial discharge date is manually input by a caregiver, such as at intake of the patient. In some examples, the initial discharge date is determined automatically—e.g., based on the intake input. For instance, the controller 108 may determine the initial discharge date based on a type of condition and/or treatment associated with the patient and/or using the algorithms described above.

At 506, the controller 108 receives information indicative of patient mobility. As described above, the information indicative of patient mobility may comprise information from one or more of the plurality of sensors within the patient environment 104, the EMR 128, third-party server(s) 134, etc.

At 508, the controller 108 determines a probability that the initial discharge date corresponds to an actual discharge date of the patient. In some examples, the actual discharge date corresponds to the estimated discharge date described above in FIG. 4. As described in FIG. 4, the probability can be determined using one or more algorithms, including a Monte Carlo analysis, a regression formula, decision tree, random forest, and/or gradient boosting machine. In some examples, the probability is determined based on information associated with the patient and/or expected conditions.

At 510, the controller 108 determines whether the probability is greater than a first threshold associated with a high confidence level. In some examples, and as described above, the first threshold is a pre-set threshold (e.g., such as 80%, 90%, or any other suitable threshold) and/or may be changed by a caregiver and/or the controller 108. In some examples, the first threshold corresponds to the confidence metric described in FIG. 4 above.

Where the controller determines the probability is greater than the first threshold (Step: 510—Yes), the method proceeds to 512. At 512, the controller 108 determines whether the probability is greater than a second threshold associated with an early discharge. In some examples, the second threshold corresponds to a threshold period of time (e.g., 8 hours, 12 hours, 24 hours, or any other suitable amount of time) that is earlier than the initial discharge date.

Where the controller 108 determines that the probability is not greater than the second threshold (Step: 512—No), the method proceeds to 514. At 514, the controller 108 determines whether there are new events requiring modification to the initial discharge date. For instance, the new events may comprise physical therapy, a new procedure and/or therapy, pre-op appointments, a new appointment (e.g., follow-up appointment with doctor, etc.), adding a practice to a patient's daily schedule (e.g., practice walking more often), etc.

Where the controller determines there are new events requiring modification to the initial discharge date (Step: 514—Yes), the method proceeds to 516. At 516, the controller 108 determines a modified discharge date. In some examples, the modified discharge date is determined based at least in part on the new events. For instance, where the new event comprises a new procedure and/or treatment, the controller 108 determines the average length of time for the procedure and/or treatment, average recovery time, etc. (based on the patient's health, age, and other factors), scheduling conflicts, etc. associated with the new event. In this example, the controller 108 determines that the new procedure results in an additional 24 hours in the healthcare facility, such that the modified discharge date is a day later than the initial discharge date. As noted above, the controller 108 may alert a caregiver of the modified discharge date, where the alert includes a request for approval of the modification. In some examples, the controller 108 modifies the discharge date based on receiving input indicating approval of the request. In other examples, the controller 108 automatically modifies the discharge date (e.g., updates the initial discharge date in the EMR 128). The method then returns to 506 and the controller 108 continues to receive information and monitor the patient 106.

Where the controller determines there are not new events requiring modification to the initial discharge date (Step: 514—No), the method returns to 506 and the controller continues to receive information and monitor the patient 106.

Where the controller determines the probability is greater than the second threshold (Step: 512—Yes), the method proceeds to 518. At 518, the controller 108 determines an early discharge date. For instance, as described above, the early discharge date is determined using one or more of a mobility progression speed and/or an events progression speed.

At 520, the controller 108 determines whether the early discharge date is a same date as the initial discharge date. For instance, where the second threshold comprises an 8-hour period of time, the controller 108 may determine that the early discharge date is the same date as the initial discharge date. Where the controller 108 determines that the early discharge date is a same date as the initial discharge date (Step: 520—Yes), the method proceeds to 522. At 522, the controller 108 generates a discharge alert. In some examples, the discharge alert includes a recommendation of an action to take with the patient (e.g., such as practicing stairs and/or walking more often). In some examples, the alert comprises an indication (e.g., such as a color) associated with the second threshold. For instance, where the second threshold is an 8-hour period of time, the alert may comprise a green indicator associated with a lower level of risk of the early discharge date being different from the initial discharge date. In some examples, as the second threshold is a larger period of time (e.g., such as 12 hours, 16 hours, etc.), the indicator may be a yellow or red indicator.

Where the controller 108 determines that the early discharge date is not the same date as the initial discharge date (Step: 520—No), the method proceeds to 524. At 524, the controller 108 updates the initial discharge date. For instance, the controller 108 sends a message to the EMR 128 to cause the EMR 128 to update the initial discharge date that is stored in the EMR 128. The method then returns to 514.

Where the controller 108 determines the probability is not greater than the first threshold (Step: 510—No), the method proceeds to 526. At 526, the controller 108 determines whether the probability is greater than a third threshold associated with a high rate of recovery. For instance, in some examples, the third threshold corresponds to a mobility progression speed and/or events progression speed described above. For instance, the third threshold may be associated with a period of time (e.g., 8-hours, 12 hours, etc.) and/or a mobility progression speed. In this example, the controller 108 determines that the patient's rate of recovery indicates that the patient is likely to be discharged on the initial discharge date, however, one or more events (indicated by an events progression speed) may be impacting the patient's overall rate of recovery and/or if the patient is on-track to be discharged by the initial discharge date.

Where the controller 108 determines the probability is greater than the third threshold (Step: 526—Yes), the method proceeds to 528. At 528, the controller 108 takes a first action. For instance, where the third threshold is 90% (e.g., indicating that it is 90% likely that the patient will be discharged on the initial discharge date), the first action taken at 528 may comprise generating an alert for a caregiver of the patient. In this example, the alert comprises an indication of one or more additional action(s) to take in order to ensure the patient is discharged on time (e.g., such as practicing walking, practicing stairs, etc.). As noted above, the alert may comprise an indicator associated with a level of action needed (e.g., such as a color).

Where the controller 108 determines the probability is not greater than the third threshold (Step: 526—No), the method proceeds to 530. At 530, the controller 108 determines whether the probability is greater than a fourth threshold associated with a medium rate of recovery. For instance, in some examples, the fourth threshold is lower than the third threshold (e.g., such as 70%). In this example, the fourth threshold indicates a lower likelihood that the patient will be discharged on the initial discharge date, due to one or more factors (e.g., event progression speed, mobility progression speed, scheduling issues, etc.).

Where the controller 108 determines the probability is greater than the fourth threshold (Step: 530—Yes), the method proceeds to 532. At 532, the controller 108 takes a second action. In some examples, the second action taken at 532 comprises generating an alert as described above. In this example, the alert may include an indicator (e.g., such as a yellow indicator), to identify an action that needs to be taken with regard to a patient. For instance, the controller 108 may determine that the patient has not met with a physical therapist, which is a required event in order to be discharged. In this example, the alert may comprise a recommendation to scheduling the meeting with the physical therapist. Additionally, or alternatively, the controller 108 may automatically schedule the meeting in response to determining the probability is greater than the fourth threshold. The method then returns to 514.

Where the controller 108 determines the probability is not greater than the third threshold (Step: 530—Yes), the method proceeds to 534. At 534, the controller 108 takes a third action. For instance, as noted above, the third threshold may be 70%. In this example, the controller 108 determines the likelihood that the patient will be discharged on the initial discharge date is less than 70%. Accordingly, the third action taken at 534 may comprise generating a recommendation to change the initial discharge date, scheduling appointment(s) for the patient, alerting caregiver(s) of the patient, alerting family member(s), and/or a social worker, etc. The method then returns to 514.

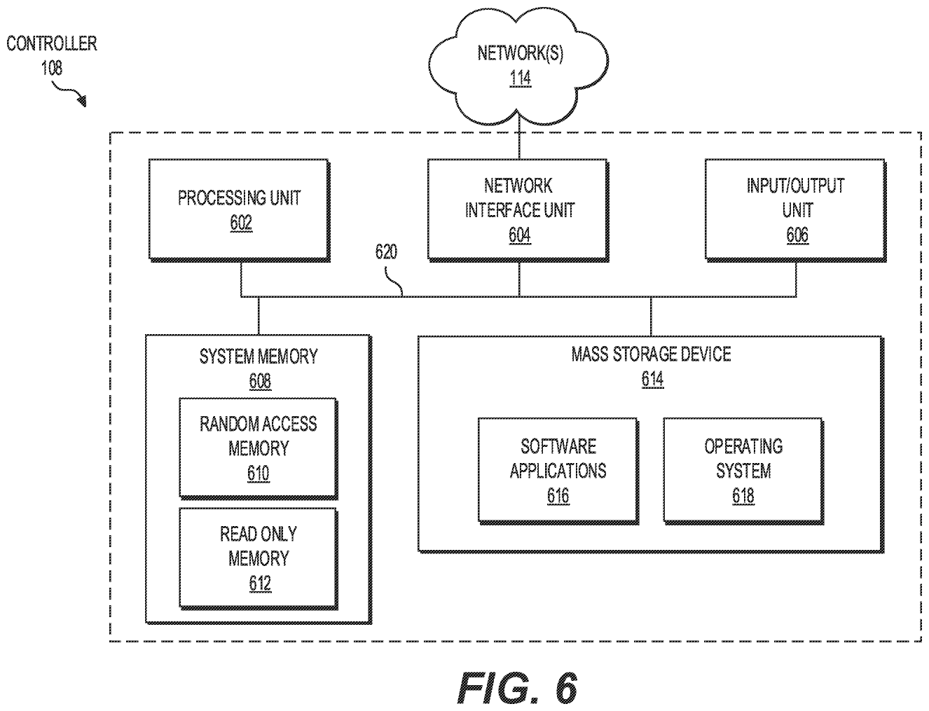

FIG. 6 schematically illustrates the controller 108 used to implement aspects of the present disclosure. The controller architecture shown in FIG. 6 illustrates any type of controller 108 and/or controller 110, and/or any type of computing device, such as a conventional server computer, workstation, desktop computer, laptop, tablet, network appliance, e-reader, smartphone, or other computing device, and can be utilized to execute any of the software components presented herein. The controller 108 may, in some examples, correspond to any device described herein, and may comprise personal devices (e.g., smartphones, tables, wearable devices, laptop devices, etc.) networked devices such as servers, switches, routers, hubs, bridges, gateways, modems, repeaters, access points, and/or any other type of computing device that may be running any type of software and/or virtualization technology.

As illustrated in FIG. 6, the controller 108 comprises a processing unit 602, a system memory 608, and a system bus 620 coupling the system memory 608 to the processing unit 602. Processing unit 602 comprises one or more processor(s), controller(s), at least one central processing unit (“CPU”), memory, and a system bus that couples the memory to the CPU. In some examples, the memory of the processing unit 602 includes system memory 608 and mass storage device. System memory 608 includes random access memory (“RAM”) 610 and read-only memory (“ROM”) 612. In some examples, a basic input/output system (BIOS) that contains the basic routines that help to transfer information between elements within the example controller 108 and/or controller 110, such as during startup, is stored in the ROM 612.

In some examples, the mass storage device 614 of the processing unit 602 stores software instructions and data. In some examples, mass storage device 614 is connected to the CPU of the processing unit 602 through a mass storage controller (not shown) connected to the system bus 620. The processing unit 602 and its associated computer-readable data storage media provide non-volatile, non-transitory storage for the example controller 108 and/or controller 110. Although the description of computer-readable data storage media contained herein refers to a mass storage device, such as a hard disk or solid state disk, it should be appreciated by those skilled in the art that computer-readable data storage media can be any available non-transitory, physical device or article of manufacture from which the central display station can read data and/or instructions.

Although the description of computer-readable data storage media contained herein refers to a mass storage device, it should be appreciated by those skilled in the art that computer-readable data storage media can be any available non-transitory, physical device or article of manufacture from which the device can read data and/or instructions. The mass storage device 614 is an example of a computer-readable storage device.

Computer-readable data storage media include volatile and non-volatile, removable and non-removable media implemented in any method or technology for storage of information such as computer-readable software instructions, data structures, program modules or other data. Example types of computer-readable data storage media include, but are not limited to, RAM, ROM, EPROM, flash memory or other solid state memory technology, CD-ROMs, digital versatile discs (“DVDs”), other optical storage media, magnetic cassettes, magnetic tape, magnetic disk storage or other magnetic storage devices, or any other medium which can be used to store the desired information and which can be accessed by the example controller 108 and/or controller 110.

The controller 108 may operate in a networked environment using logical connections to remote network devices, including the mobile devices 126, EMR 128, nurse call server 130, nurses' station 132, and/or third-party server(s) 134, through the network(s) 114. The controller 108 connects to the network(s) 114 through a network interface unit 604 connected to the system bus 620. The network interface unit 604 may also be utilized to connect to other types of networks and remote computing systems.

Input/output unit 606 is configured to receive and process input from a number of input devices. Similarly, the input/output unit 606 may provide output to a number of output devices.

Mass storage device 614 and/or RAM 610 store software instructions and data. For instance, the software instructions can include an operating system 618 suitable for controlling the operation of a device. The mass storage device 614 and/or the RAM 610 also store software instructions 616, that when executed by the processing unit 602, cause the device to perform the techniques described herein.

As a result, the methods and systems described herein may assist caregivers with patient care and reduce day of discharge surprises. Additionally, by continuously monitoring patient mobility, event progression, etc., the techniques and systems described herein enable healthcare facilities to provide personalized care to patients. This may streamline workflow for providing care within the healthcare facility, thereby reducing costs for the patient and/or the healthcare facility.

The foregoing is merely illustrative of the principles of this disclosure and various modifications can be made by those skilled in the art without departing from the scope of this disclosure. The above described examples are presented for purposes of illustration and not of limitation. The present disclosure also can take many forms other than those explicitly described herein. Accordingly, it is emphasized that this disclosure is not limited to the explicitly disclosed methods, systems, devices, and apparatuses, but is intended to include variations to and modifications thereof, which are within the spirit of the following claims.

As a further example, variations of apparatus or process limitations (e.g., dimensions, configurations, components, process step order, etc.) can be made to further optimize the provided structures, devices, and methods, as shown and described herein. In any event, the structures and devices, as well as the associated methods, described herein have many applications. Therefore, the disclosed subject matter should not be limited to any single example described herein, but rather should be construed in breadth and scope in accordance with the appended claims.

In some instances, one or more components may be referred to herein as “configured to,” “configurable to,” “operable/operative to,” “adapted/adaptable,” “able to,” “conformable/conformed to,” etc. Those skilled in the art will recognize that such terms (e.g., “configured to”) can generally encompass active-state components and/or inactive-state components and/or standby-state components, unless context requires otherwise.

The description and illustration of one or more embodiments provided in this application are not intended to limit or restrict the scope of the invention as claimed in any way. Regardless whether shown and described in combination or separately, the various features (both structural and methodological) are intended to be selectively included or omitted to produce an embodiment with a particular set of features. Having been provided with the description and illustration of the present application, one skilled in the art may envision variations, modifications, and alternate embodiments falling within the spirit of the broader aspects of the claimed invention and the general inventive concept embodied in this application that do not depart from the broader scope.

What is claimed is:

1. A method, comprising:
receiving an input indicating intake information associated with a patient;
determining, based on the input, an initial discharge date;
receiving mobility information associated with the patient;
determining, based at least partly on the mobility information, an estimated discharge date and a confidence metric associated with the estimated discharge date;
determining that the estimated discharge date is later than the initial discharge date by more than a threshold period of time;
determining that the confidence metric is greater than a threshold metric; and
based at least partly on the estimated discharge date being later than the initial discharge date by more than the threshold period of time and the confidence metric being greater than the threshold metric, generating an alert.

2. The method of claim 1, further comprising:
receiving additional mobility information from an electronic health record associated with the patient; and
determining the estimated discharge date and the confidence metric based at least partly on the additional mobility information.

3. The method of claim 1, wherein the mobility information is received from a plurality of sensors, a camera, or a mobility assistance device.

4. The method of claim 1, further comprising
transmitting the alert to an electronic device via a network, the alert comprising a selectable option to schedule an appointment for the patient and causing a user interface of the electronic device to display the alert.

5. The method of claim 4, wherein the input comprises a first input, the method further comprising:
receiving, from the electronic device, a second input indicating acceptance of the selectable option; and
scheduling, based at least in part on the second input, the appointment for the patient.

6. The method of claim 1, wherein the alert comprises an indication to change the initial discharge date to a new discharge date, the method further comprising:
transmitting a message to a remote server to cause the initial discharge date to be updated to the new discharge date.

7. The method of claim 1, wherein the alert includes a visual indicia associated with one or more of the threshold period of time or the confidence metric, the visual indicia comprising at least a first color associated with the threshold period of time or a second color associated with the confidence metric.

8. The method of claim 1, wherein the one or more threshold metrics comprises a first threshold metric and a second threshold metric, the method further comprising:
determining that the estimated discharge date is later than the initial discharge date by more than a second threshold period of time;
determining that the confidence metric is greater than the second threshold metric; and
based at least in part on determining that the estimated discharge date is later by more than the second threshold period of time and the confidence metric is greater than the second threshold metric, generating the alert.

9. A system, comprising:
a network;
a computing device connected to the network;
a controller in communication with the computing device via the network;
a plurality of sensors operably connected to the controller via the network; and
one or more non-transitory computer-readable media storing instructions that, when executed by the controller, cause the controller to perform operations comprising:
receive, via the network and from the computing device, an input indicating intake information associated with a patient;
determine, based on the input, an initial discharge date;
receive, via the network and from the plurality of sensors, mobility information associated with the patient;
determine, based at least partly on the mobility information, an estimated discharge date;
determine that the estimated discharge date is later than the initial discharge date by more than a threshold period of time;
generate an alert based at least partly on the estimated discharge date being later than the initial discharge date by more than the threshold period of time; and
provide, via the network, the alert to the computing device, the alert causing the computing device to display at least one of a visual indicia or a suggestion in response to receiving the alert.

10. The system of claim 9, the operations further comprising:
determine, based at least partly on the mobility information, a confidence metric associated with the estimated discharge date;
determine that the confidence metric is greater than a threshold metric;
determine that the estimated discharge date is earlier than the initial discharge date by more than a second threshold period of time;
determine that the confidence metric is greater than a second threshold metric; and
wherein the controller generates the alert based at least in part on determining that the estimated discharge date is earlier by more than the second threshold period of time and the confidence metric is greater than the second threshold metric.

11. The system of claim 10, wherein the suggestion is to change the initial discharge date to an earlier date.

12. The system of claim 10, the operations further comprising:
send, via the network, the alert to the computing device, the computing device being associated with at least one of a caregiver, a nurse, or a doctor, wherein the alert causes the computing device to display a suggestion to change the initial discharge date to an earlier date; and
cause the initial discharge date to be updated to the earlier date based at least in part on sending the alert.

13. The system of claim 9, the operations further comprising:
determine, based at least partly on the mobility information, a confidence metric associated with the estimated discharge date;
determine that the confidence metric is greater than a threshold metric; and
generate the alert based at least partly on the confidence metric being greater than a threshold metric, wherein the visual indicia is associated with one or more of the threshold period of time or the confidence metric, the visual indicia comprising at least a first color associated with the threshold period of time or a second color associated with the confidence metric.

14. The system of claim 9, wherein the plurality of sensors comprise one or more cameras, motion sensors, GPS sensors, accelerometers, and real-time locating system tags.

15. A method comprising:
determining, by a controller, one or more of an initial discharge date, a mobility requirement, or a discharge event;
receiving, by the controller, mobility information associated with a patient;
determining, by the controller and based at least partly on the mobility information, an estimated discharge date and a confidence metric associated with the estimated discharge date;
determining, by the controller, that at least one of the mobility requirement or the discharge event is achievable by a date that is later than the initial discharge date;
determining, by the controller, that the confidence metric is greater than a threshold metric; and
generating, by the controller, an alert.

16. The method of claim 15, wherein determining the at least one of the mobility requirement or the discharge event is achievable by date that is later than the initial discharge date is based at least in part on a target discharge location associated with the patient.

17. The method of claim 15, wherein the mobility information is received from one or more of a plurality of sensors, a camera, or a mobility assistance device.

18. The method of claim 15, further comprising:
determining that the confidence metric is greater than a second threshold metric; and
based at least in part on determining that the confidence metric is greater than the second threshold metric, generating the alert; and
sending the alert to a device of at least one of a caregiver, a nurse, a social worker, or a doctor.

19. The method of claim 18, wherein the alert comprises an indication of one or more of whether to add an action to a schedule of the patient, whether a target location needs to change, or whether the initial discharge date needs to change.

20. The method of claim 15, further comprising:
receiving additional mobility information from an electronic health record associated with the patient; and
determining the estimated discharge date and the confidence metric further based at least partly on the additional mobility information.
